(19) United States
(12) Patent Application Publication
(10) Pub. No.: US 2014/0024621 A1
(43) Pub. Date: Jan. 23, 2014

(54) AMINOPYRIDINE COMPOUNDS AND THEIR USES

(71) Applicants: Michael J. Hudson, Berkshire (GB); Alan Palmer, Berkshire (GB); Richard Todd, Buckinghamshire (GB); Patrick Camilleri, Herts (GB)

(72) Inventors: Michael J. Hudson, Berkshire (GB); Alan Palmer, Berkshire (GB); Richard Todd, Buckinghamshire (GB); Patrick Camilleri, Herts (GB)

(73) Assignee: MS THERAPEUTICS LIMITED, Berkshire (GB)

(21) Appl. No.: 13/555,720

(22) Filed: Jul. 23, 2012

(51) Int. Cl. A61K 31/44; A61K 31/4409; C07F 9/58; A61K 31/675; A61P 25/00; A61K 31/4433; A61K 31/655; C07D 213/74; C07D 213/76; C07D 213/75; C07D 405/12

(52) U.S. Cl. 514/89; 546/308; 514/353; 514/352; 546/22; 546/282.1; 514/336; 534/766; 514/150; 546/307

(57) ABSTRACT

The invention generally relates to aminopyridines and methods of use thereof. In certain embodiments, the invention provides an aminopyridine or a pharmaceutically-acceptable salt thereof, in which the aminopyridine or the salt thereof includes a cleavable functional group that substantially prevents extra-hepatic hydrolysis.

### FIELD OF THE INVENTION

The invention generally relates to aminopyridines and methods of use thereof.

### BACKGROUND

Neurons are the basic cell of the brain and nervous system. By transmitting signals to and from the brain and throughout the body, neurons coordinate a body's actions and functions. Within a neuron, signals are transmitted as electrochemical impulses along fibers called axons and between neurons and between neurons and other tissues (mainly muscle) the impulse is usually mediated by the depolarization-evoked release of neurotransmitter. This is accomplished, in-part, through the action of a series of potassium channels across the neuronal cell membrane. In a resting state, a cell membrane is polarized due to higher concentrations of sodium ions outside than inside the neuron and higher concentrations of potassium ions inside than outside the neuron. Arrival of a signal causes a temporary depolarization of a region of the membrane. This depolarization is caused by the transient opening of sodium channels and an influx of sodium ions. The depolarized region (the action potential) then progresses down the axon, thereby transmitting the electrical signal to the nerve terminal from which neurotransmitter is released to permit the signal to be transferred to another neuron or to a muscle. The depolarized region is subsequently re-polarized by opening of voltage-gated potassium channels and the efflux of potassium ions. The potassium channels then close. After the potassium channels are closed, ion pumps restore the original sodium and potassium ion concentration. The neuron is thereby returned to its resting (polarized) state, and is available to transmit another signal along the axon.

Certain neural disorders arise when tissue damage, disease, or chemicals interfere with ability of a neuron to transmit a signal. Exemplary neural disorders include demyelinating diseases, neurodegenerative diseases, traumatic brain and spinal cord injury, neuropathies, neuromuscular diseases, and poisoning by neuromuscular blocking agents. Damage to or dysfunction of nerve tissue can inhibit or diminish successful signal transmission. Multiple sclerosis, for example, causes damage to the myelin sheath that surrounds axons. It provides electrical insulation for the axon by reducing ion leakage and thus decreasing the capacitance of the axonal membrane. Myelin also increases signal speed since it permits saltatory propagation of action potentials between the numerous small areas along the axon (the nodes of Ranvier) that are not surrounded by myelin.

Aminopyridines are a class of compounds that block potassium channels as exemplified by 4-aminopyridine (H₂C₅H₄N), a central nervous stimulant that has recently been licensed for human therapeutic use as well as having a long history of veterinary use to reverse the effects of certain anesthetics and sedatives as well as being used as a pest bird flock deterrent. By blocking the transient efflux of potassium through voltage-gated potassium channels along the axon or at a nerve terminal or both, aminopyridines prolong the action potential and thus can improve signal conduction in damaged or dysfunctional nerves. Accordingly, aminopyridines are potentially valuable for treating diseases, disorders or conditions associated with impaired or diminished signal transmission in neurons.

However, a problem associated with the clinical use of aminopyridines is their potential to cause seizures as a consequence of movement through the blood-brain barrier into the interstitial fluid of the brain parenchyma where, once a sufficient concentration is achieved, they can over-stimulate brain neurons. This means that such compounds often have a low therapeutic index, which is defined as the dose causing side-effects/the dose required for therapeutic efficacy.

Other adverse side effects include nausea, dizziness, and respiratory failure. Due to those adverse side effects, aminopyridines are of limited use in treating neural disorders.

One approach to avoiding unacceptable brain concentrations of aminopyridines involves delivering these compositions at low concentrations. However, this approach simply limits the clinical use to restricted doses and fails to provide meaningful control over the resulting brain concentration. In another approach, aminopyridine compositions are infused directly into the spinal fluid to treat spinal injuries. However, spinal infusion is problematic because it is highly invasive, requiring complex surgery that involves insertion of a cannula into the spinal cord. Thus, while aminopyridines show promise for treating neural disorders, the inability to control the relative plasma and brain concentrations of these compounds has limited their widespread clinical use.

### SUMMARY

The invention provides a means for delivering aminopyridine compounds to humans and animals that does not lead to their accumulation in the brain upon administration of therapeutic doses and therefore provides new aminopyridine compounds with an improved therapeutic index. In this manner, compounds of the invention provide beneficial pharmaceutical properties for treating neural disorders without producing the harmful side effects that are generally associated with this class of compounds. To avoid accumulation in the brain, compounds of the invention are formulated with at least one cleavable functional group that substantially inhibits gastrointestinal hydrolysis of the aminopyridine and provides for targeted metabolic biotransformation of the compound in the liver to generate an active agent. Accordingly, the aminopyridines of the invention exhibit a slow rate of gastrointestinal hydrolysis and a good rate of penetration into the liver. In this manner, relative plasma and brain concentrations are controlled, allowing for aminopyridine compounds of the invention to be used to treat neural disorders while avoiding adverse side effects associated with this class of compounds.

Compositions of the invention include any compounds that result in an active aminopyridine being produced within the body upon cleavage of the functional group that controls delivery of the compound to the liver. Aminopyridines of the invention include, for example, aminopyridine or diaminopyridine, particularly 4-aminopyridine, 3,4-diaminopyridine, 2,4-diaminopyridine and 3,4,5-triaminopyridine. In certain embodiments, the cleavable functional group includes, for example, an amino acid, an alkyl group, a pyrone, a phosphonic or sulfamic acid, or an acyloxyalkylcarbamate. The cleavable functional group is attached, for example, to the nitrogen of an amino group. The attachment can be, for example, in the amide, imine, carbamate, enamine, or azo form.

In certain aspects, the invention provides an aminopyridine or a pharmaceutically-acceptable salt thereof, in which the aminopyridine or the salt thereof includes a cleavable functional group that substantially prevents hydrolysis outside of the liver. In certain embodiments, the aminopyridine has a pKa that ranges from about 4.9 to about 8.2 and a logP that ranges from about 0.8 to about 2.3.

In certain embodiments the aminopyridine is represented by formula (I):

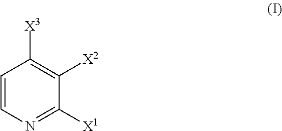

in which X¹ and X² are each independently selected from H, NH₂, NHR¹, N═NR², or N═R³; X³ is selected from NH₂, NHR¹, N═NR², or N═CR³; R¹ is selected from: COR⁴, where R⁴ is an amino acid attached at the carbonyl carbon; COOR⁵, where R⁵ is an alkyl group; COR⁶, where R⁶ is a branched chain alkyl optionally substituted with a phenyl group, and the phenyl group is optionally substituted with a phosphonooxy group and optionally substituted with one or more alkyl groups; a pyrone; and SO₃Na; R² is a heterocycloalkenyl optionally substituted with one or more amino groups; R³ is a phenyl group optionally substituted with one or more halogens; or R³ is represented by one of formulas (II), (III), (IV), (V), (VI), (VII), (VIII), and (IX):

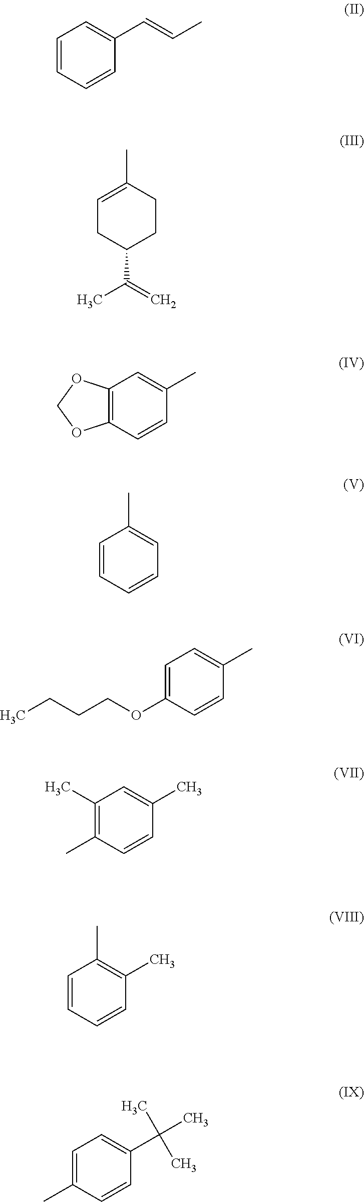

with the proviso that when X³ is COOR⁵, R⁵ is not a methyl group, ethyl group, tert-butyl group, or n-dodecyl group.

In certain embodiments R⁴ is alanine, lysine, or phenylalanine. The invention further provides compounds of formula (II), in which the aminopyridine is represented by one of formulas (X), (XI), (XII), (XIII), (XIV), (XV), (XVI), (XVII), and (XVIII):

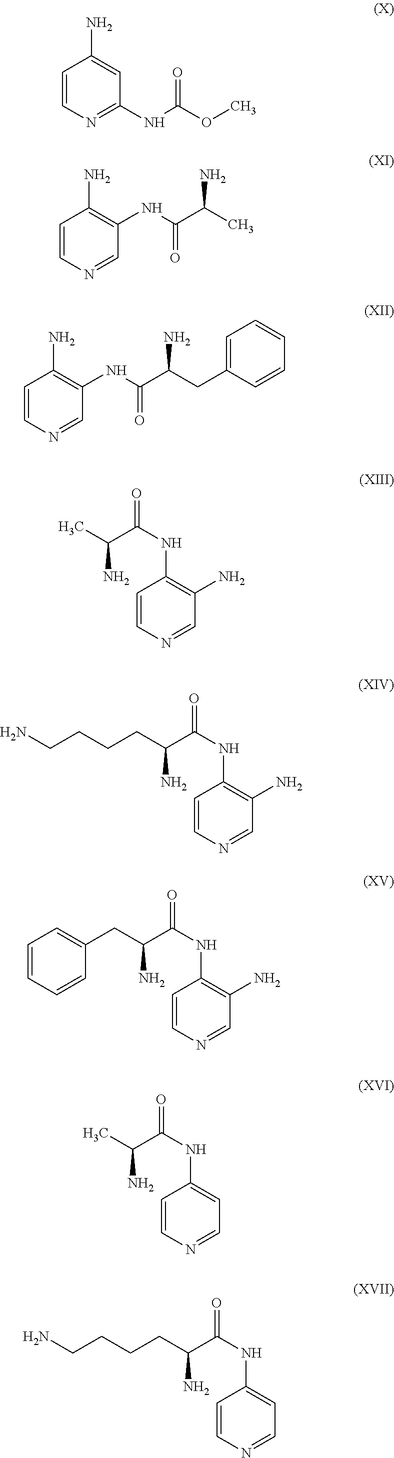

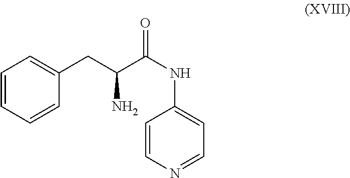 [SMILES: N[C@@H](Cc1ccccc1)C(=O)Nc1ccncc1]

In certain embodiments, the aminopyridine is represented by formula (XIX):

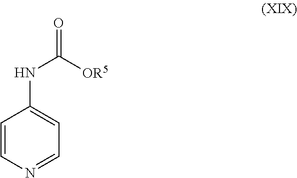

in which R⁵ is an n-propyl group; an n-butyl group; a sec-butyl group, or a straight or branched C₅ or higher alkyl chain, with the proviso that R⁵ is not an n-dodecyl group.

In certain embodiments, the present invention further provides an aminopyridine represented by one of formulas (XX), (XXI), (XXII), and (XXIII):

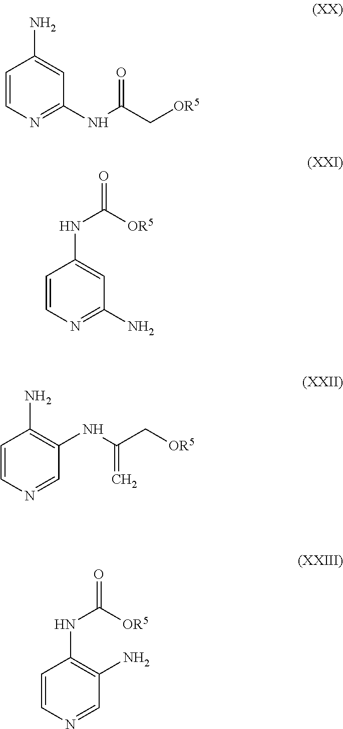

in which R⁵ is an alkyl group.

The invention further provides an aminopyridine represented by any of formulas (XXIV), (XXV), (XXVI), (XXVII), and (XXVIII):

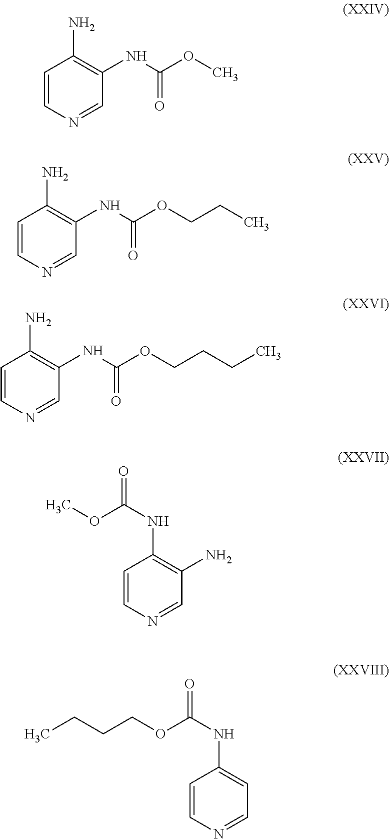

In certain embodiments, the invention provides an aminopyridine represented by one of formulas (XXIX), (XXX), and (XXXI):

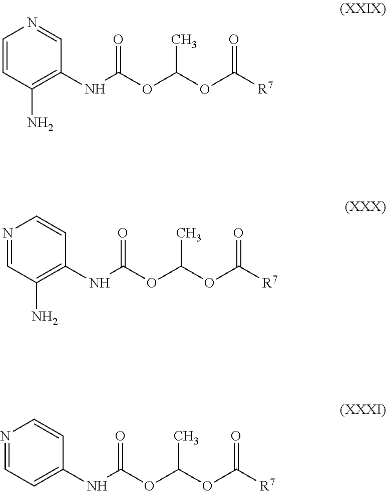

In which R⁷ is a alkyl chain. In certain embodiments, R⁷ is a C₁-C₆ straight or branched-chain alkyl.

In certain embodiments, the invention provides an aminopyridine represented by formula (II), in which R⁶ is represented by formula (XXXII):

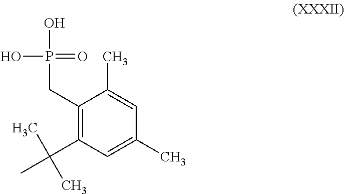 [SMILES: Cc1cc(C)c(CP(=O)(O)O)c(C(C)(C)C)c1]

In certain embodiments, the invention provides an aminopyridine represented by the formula (XXXIII):

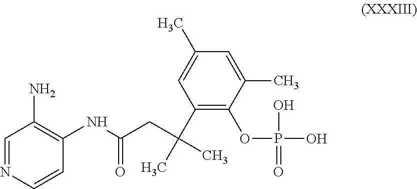 [SMILES: Cc1cc(C)c(OP(=O)(O)O)c(C(C)(C)CC(=O)Nc2ccncc2N)c1]

In certain embodiments, the invention further provides an aminopyridine represented by formula (II), in which X³ is NH₂, X¹ is H, and X² is N═R³. In certain embodiments, the aminopyridine is represented by formula (XXXIV):

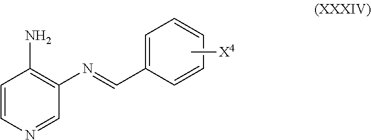

in which X⁴ is a halogen.

In certain embodiments, the aminopyridine is represented by one of formulas (XXXV), (XXXVI), and (XXXVII):

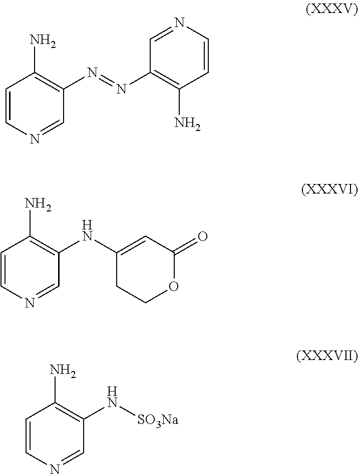

In certain aspects, the invention provides a method of treating a neural disorder that involves administering an effective dose of an aminopyridine, or a pharmaceutically acceptable salt thereof, in which the aminopyridine, or the salt thereof, includes a cleavable functional group that substantially prevents extra-hepatic hydrolysis of the aminopyridine. Exemplary neural disorders include a neuropathy, a neuromuscular disorder, or a poisoning by a neuromuscular blocking agent.

### DETAILED DESCRIPTION

The invention generally relates to aminopyridine compounds that do not accumulate in the brain upon administration to a person. In certain embodiments, aminopyridines of the invention are formulated with at least one cleavable functional group that substantially inhibits extra-hepatic (gastrointestinal) hydrolysis of the aminopyridine and provides for targeted hepatic (liver) hydrolysis of the compound. Accordingly, the invention provides an aminopyridine or a pharmaceutically-acceptable salt thereof, in which the aminopyridine or the salt thereof includes a cleavable functional group that substantially prevents extra-hepatic hydrolysis.

The relative plasma and brain concentrations of the active aminopyridine are dependent on the rate of extra-hepatic hydrolysis of the administered aminopyridine. Compounds that exhibit only slow extra-hepatic hydrolysis exhibit good rate penetration into the liver. The rate of penetration and the location of hydrolysis are therefore two parameters of interest relating to the adsorption, distribution, metabolism, and excretion (ADME) of aminopyridines.

The ADME of aminopyridines is influenced by their extent of binding to plasma proteins, rates of irreversible metabolism, octanol/water partition coefficient constant (logP), partition coefficient at a particular pH (logD), fractional charges at physiological pH, and acid dissociation constant (pKa). For example, the rate of hepatic penetration is strongly controlled by logD. The logD for an aminopyridine compound relates to the logP and pKa values for that compound. The choice of a cleavable functional group provides control over a compound's logP and pKa.

In one embodiment, aminopyridines of the invention have a pKa and a logP value conferring on the compound a good rate of hepatic penetration. Such aminopyridines exhibit a slow rate of extra-hepatic hydrolysis. Aminopyridine compounds with a slow rate of extra-hepatic hydrolysis and an increased rate of hepatic penetration exhibit plasma selectivity.

One of skill in the art will be able to select an appropriate cleavable functional group based on these considerations. The cleavable functional group may be used to target the compound for a particular rate of extra-hepatic hydrolysis. In one embodiment, extra-hepatic hydrolysis occurs at a rate that is fast (about 0.1/min), medium (about 0.01/min), slow (about 0.001/min), or zero. In one embodiment, the cleavable functional group is used to target the aminopyridine for a slow rate of extra-hepatic hydrolysis. In one embodiment, the aminopyridine has a pKa range from about 4.9 to about 8.2 and a logP range from about 0.8 to about 2.3.

Hepatic hydrolysis reduces the concentration and residency time of the active aminopyridine in the brain (brain AUC) while maintaining efficacious plasma concentrations (plasma AUC). Thus, compounds of the invention exhibit selectivity for plasma versus brain for the active aminopyridine.

The plasma selectivity of an aminopyridine is evaluated by determining the brain AUC and plasma AUC resulting from administration of a compound of the invention in the uncleaved form and comparing these to a brain AUC and plasma AUC resulting from an administration of the active aminopyridine, i.e., in the cleaved form. The plasma selectivity of an aminopyridine of the invention is represented by A:

$$A = \frac{( AUCbrain / AUCplasma ) uncleaved}{( AUCbrain / AUCplasma ) cleaved}$$

The plasma selectivity of an aminopyridine is also evaluated by determining the brain Cmax and plasma Cmax resulting from administration of a compound of the invention in the uncleaved form and comparing these to a brain Cmax and plasma Cmax resulting from an administration of the active aminopyridine, i.e., in the cleaved form. The plasma selectivity of an aminopyridine of the invention is represented by B:

$$B = \frac{[ \frac{Cmax ( brain )}{Cmac ( plasma )} ] uncleaved}{[ \frac{Cmax ( brain )}{Cmac ( plasma )} ] un cleaved !}$$

Aminopyridines with A>1 or B>1 exhibit greater selectivity for plasma versus brain. In one embodiment, aminopyridines of the invention have an A range from about 1.13 to about 2.02.

Data herein demonstrate that the rate of extra-hepatic hydrolysis is an important factor for determining plasma and brain levels of the free active aminopyridine. A slow rate of extra-hepatic hydrolysis results in A>1. The cleavable functional group attached to compounds of the invention inhibits extra-hepatic hydrolysis, thereby targeting compounds of the invention for hepatic hydrolysis. Thus, through the choice of the cleavable functional group, relative plasma and brain levels of the active aminopyridine are modulated.

In certain embodiments, aminopyridines of the invention exhibit slow extra-hepatic hydrolysis, fast hepatic hydrolysis, and no hepatic inactivation. In certain embodiments, aminopyridines of the invention exhibit a plasma selectivity of A=1.13, A=1.21, A=1.40, or A=2.02.

Cleavage of the functional group converts NH to NH₂ and produces an active aminopyridine. Any compound that results in an active aminopyridine within the body upon cleavage of the functional group is envisioned and within the scope of the invention. A number of aminopyridines, including mono-, di- and tri-aminopyridines such as 4-aminopyridine (4-AP), 3,4-diaminopyridine (3,4-DAP) and 3,4,5-triaminopyridine (3,4,5-TAP), block voltage-dependent potassium channels in both vertebrate and invertebrate tissues. In certain embodiments, the invention provides an active agent that includes at least one of 4-AP, 3,4-DAP, and 2,4-diaminopyridine (2,4-DAP). Aminopyridines of the invention further include cleavable functional groups attached to either one or two of the amino groups. In certain embodiments, the cleavable functional groups can include amides, including natural and unnatural amino acids, carbamates, and phosphonic acids.

In certain embodiments, the invention provides aminopyridine compounds that have a cleavable functional group that include, for example, compounds represented by formulas (XXXVII), (XXXVIII), (XXXIX), (XL), and (XLI).

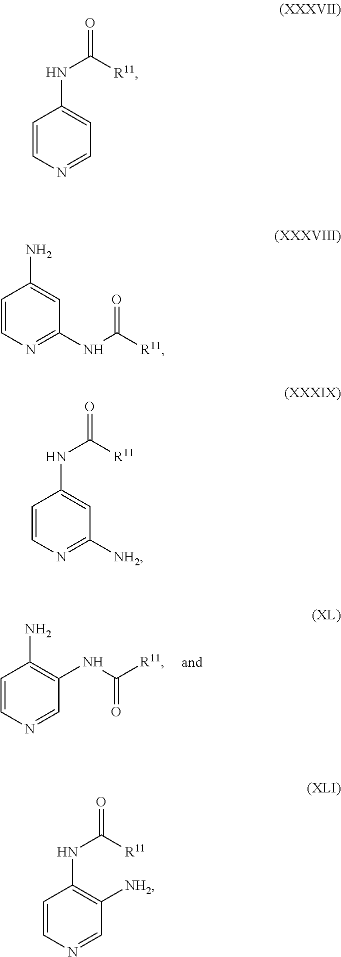

in which R¹¹ is an amino moiety, attached through the carboxyl group.

In certain embodiments, the invention provides an aminopyridine having a pKa that ranges from about 4.9 to about 8.2, a logP that ranges from about 0.8 to about 2.3, and exhibiting A in a range from about 1.13 to 2.02. In certain embodiments, the invention provides an aminopyridine with a pKa=8.2, a logP=1.7, and exhibiting A=1.13 An exemplary aminopyridine with a cleavable functional group having these properties is represented by formula (XXV):

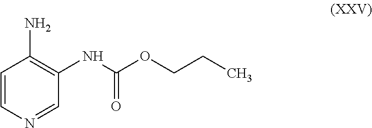

In certain embodiments, the invention provides an aminopyridine with a pKa=8.2, a logP=2.3, and exhibiting A=2.02. An exemplary aminopyridine with a cleavable functional group having these properties is represented by formula (XXVI):

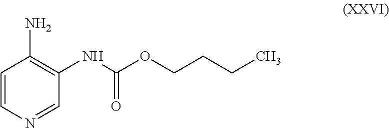

In certain embodiments, the invention provides an aminopyridine with a pKa=5.4, a logP=0.8, and exhibiting A=1.21. An exemplary aminopyridine with a cleavable functional group having these properties is represented by formula (XXVII):

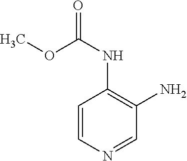

In certain embodiments, the cleavable functional group is a phosphonic acid, for example as represented by formula (XXXII):

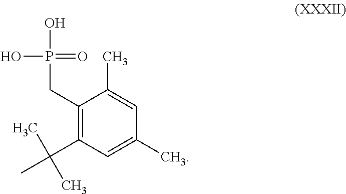 [SMILES: Cc1cc(C)c(CP(=O)(O)O)c(C(C)(C)C)c1]

In some embodiments, the invention provides an aminopyridine including a carbamate. Typical carbamate aminopyridines with a cleavable functional group include alkylcarbamates, for example as represented by formulas (XIX), (XX), (XXI), (XXII), and (XXIII):

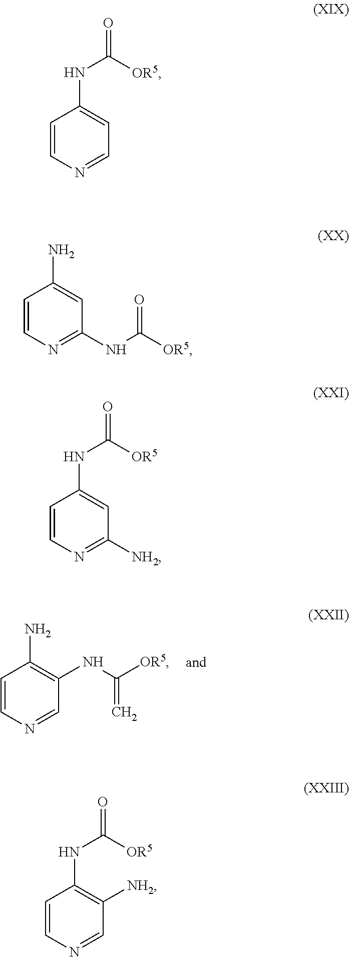

in which R⁵ is an alkyl group.

In some embodiments, the invention provides acyloxyalkylcarbamates of aminopyridines with a cleavable functional group, for examples, as represented by formulas (XXIX), (XXX), and (XXXI):

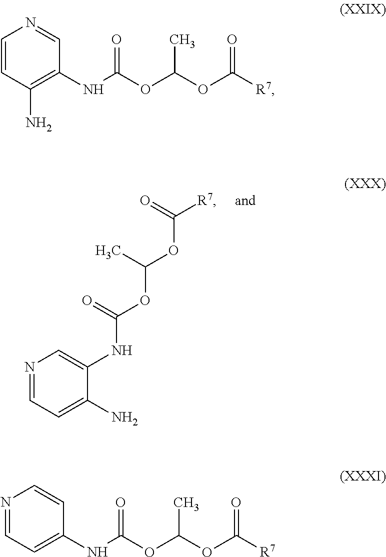

in which R⁷ is a alkyl chain. In certain embodiments, R⁷ is a C₁-C₆ straight or branched-chain alkyl.

In certain embodiments, the invention provides an aminopyridine with a cleavable functional group including an azo functional group including, for example, R—N═N—R′. For example, in certain embodiments, the invention provides an aminopyridine with a cleavable functional group with an azo group represented by formula (XXXV):

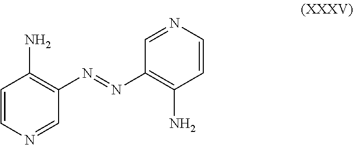 [SMILES: Nc1ccncc1/N=N/c1cnccc1N]

In certain embodiments, the invention provides an aminopyridine with a cleavable functional group including an enamine. One skilled in the art will recognize that an enamine can be obtained by reacting an aldehyde with the amino group of an aminopyridine. One such enamine is represented by formula (XXXVI):

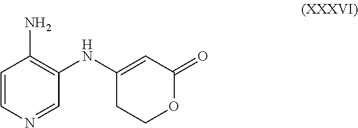 [SMILES: Nc1ccncc1NC1=CC(=O)OCC1]

In certain embodiments, the invention provides an aminopyridine with a cleavable functional group including a sulfamic acid sodium salt, for example, as represented by formula (XXXVII):

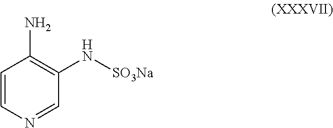

In certain embodiments, the invention provides an aminopyridine with a cleavable functional group including an imine functional group in which an amino nitrogen participates in a double bond to a carbon. Aminopyridines of the invention with an imine functional group can be made by the reaction of aminopyridine with an aldehyde.

Aldehydes that can be used for the formation of aminopyridines of the invention include: cinnamaldehyde, formula (XLII); perillaldehyde, formula (XLIII); piperona, formula (XLIV); benzaldehyde, formula (XLV); 4-butoxybenzaldehyde, formula (XLVI); 3,4-dimethylbenzaldehyde, formula (XLVII); salicylaldehyde, formula (XLVIII) and 4-tert-butylbenzaldehyde, formula (XLIX):

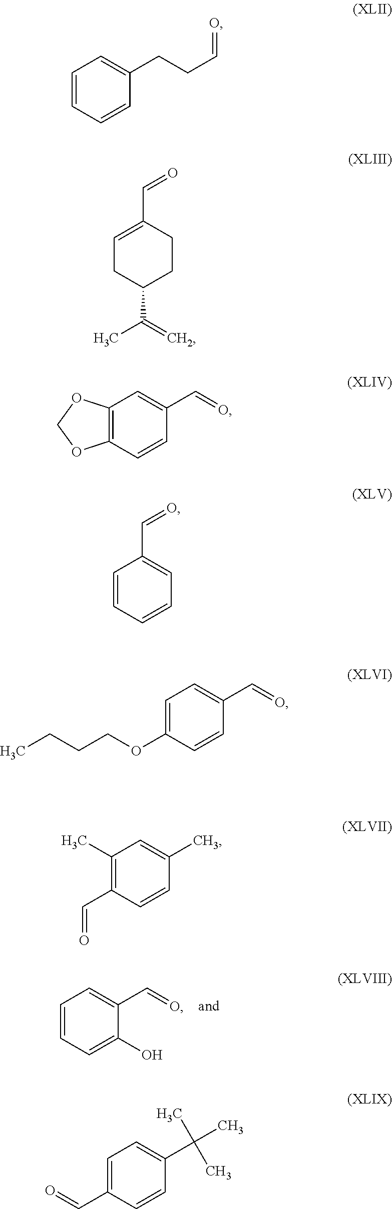

In certain embodiments, the invention provides an aminopyridine with a cleavable functional group including an imine functional group, for example as represented by formula (XXXIV):

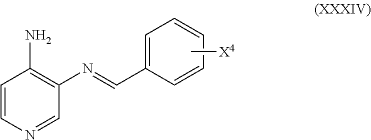

in which X⁴ is a halogen.

Shown below are exemplary synthesis routes to obtain compounds of the invention. For example, in some embodiments, reaction of the aminopyridine with a suitable intermediate produces the desired aminopyridine. Is some embodiments, the cleavable functional group can be attached to a particular amino group of an aminopyridine by first protecting another amino group in a protecting step, then reacting the protected aminopyridine with a suitable intermediate, and then de-protecting the product of that reaction in a de-protecting step.

In some embodiments, aminopyridines of the invention include acyloxyalkylcarbamate esters of aminopyridine, in which an acyloxyalkylcarbamate cleavable functional group is bound to an amino nitrogen of either 3,4-DAP, 2,4-DAP, or 4-AP. Provided below are synthetic pathways resulting in an acyloxyalkylcarbamate cleavable functional group at either a 3-amino group or a 4-amino group of 3,4-DAP or at the 4-amino group of 4-AP.

For example, a synthetic pathway is shown which provides 3,4-DAP including an acyloxyalkylcarbamate cleavable functional group at the 3-amino group as represented by formula (XXIX):

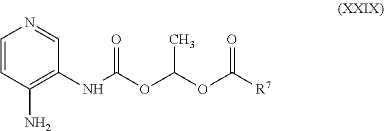

in which R⁷ is a alkyl chain such as a C₁-C₆ straight or branched-chain alkyl.

A compound represented by formula (XXIX) can be prepared by three potential routes (Paths A-C below). Paths A and B proceed via the same intermediate thiocarbonate (L), synthesized in two steps from 1-chloroethyl chloroformate. Such general chemistry is described in U.S. Pat. No. 5,401,868 and PCT Publication WO 2010/008886, both herein incorporated by reference in their entireties. Path C proceeds via the 4-nitrophenyl carbonate (LII) and the acyloxy carbonate (LIII) and is described in Alexander, et al., J. Med. Chem. 1988, 31:318-322, herein incorporated by reference in its entirety. Selection of either Paths A, B or C would be based on several criteria: the relative stability of intermediates, physical properties of the intermediates and reactivity of intermediates (L), (LII) and (LIII) towards 3,4-DAP.

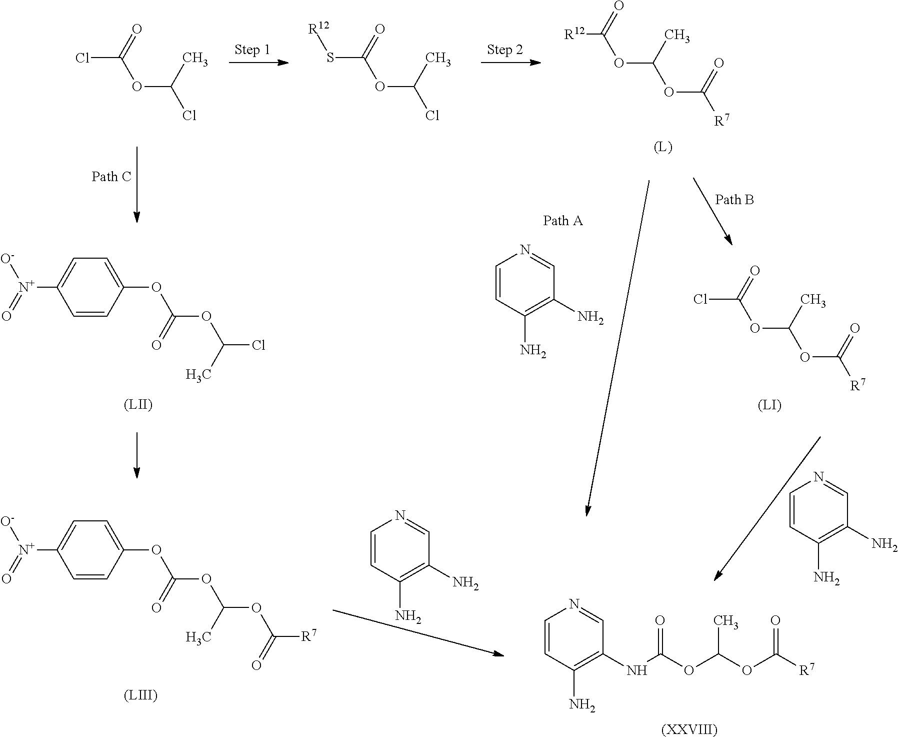

in which R¹² and R⁷ are alkyl chains such as a C₁-C₆ straight or branched-chain alkyl.

In another example, a synthetic pathway is provided below, which provides 3,4-DAP including an acyloxyalkylcarbamate cleavable functional group at the 4-amino group as represented by formula (XXX):

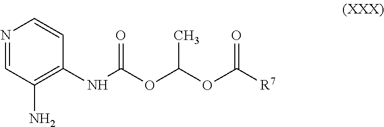

in which R⁷ is a alkyl chain such as a C₁-C₆ straight or branched-chain alkyl.

A compound represented by formula (XXX) could be prepared by a synthesis route in which an acyloxyalkyl carbamate side chain is attached to the 4-amino group of 3,4-DAP. Such compounds are prepared by selecting an appropriate protection/de-protection strategy, as below:

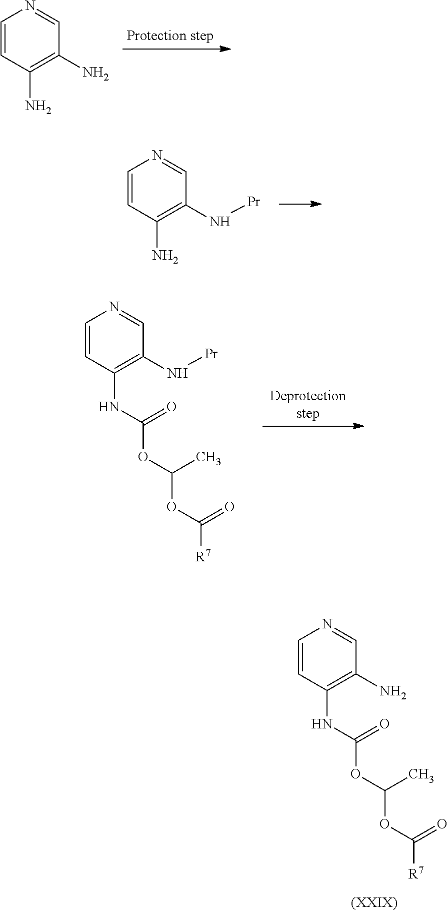

where Pr is a protecting group and R⁷ is a straight or branched chain alkyl.

In a further example, a synthetic pathway is given below, which yields 4-AP including an acyloxyalkylcarbamate cleavable functional group at the amino group as represented by formula (XXXI):

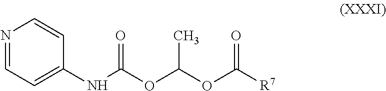

in which R⁷ is a alkyl chain such as a C₁-C₆ straight or branched-chain alkyl.

Compounds having formula (XXXI) can be prepared from 4-AP. Selection of the preferred route to provide (7) would be dictated by the same criteria as for 3,4-DAP.

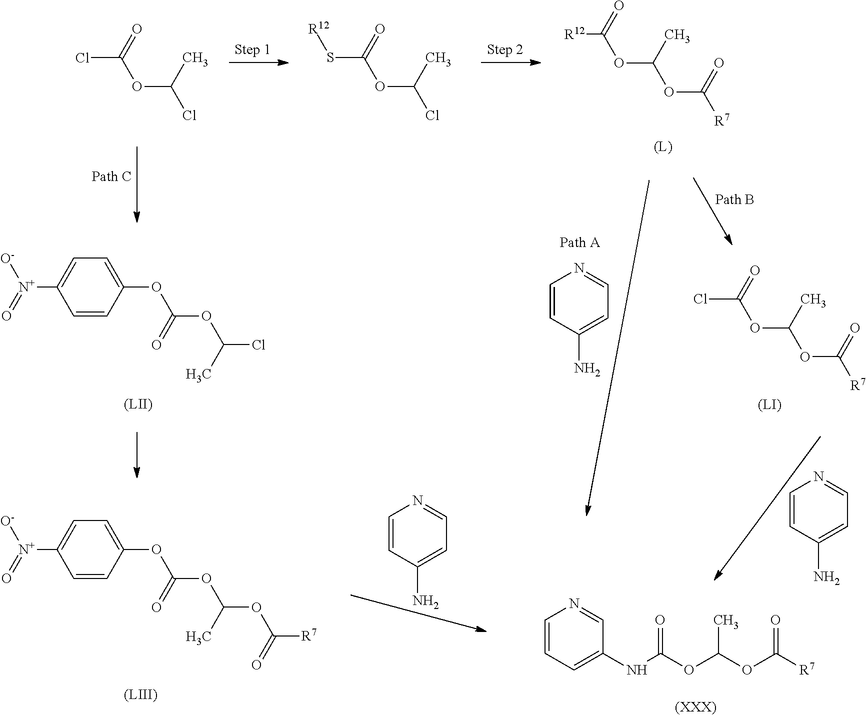

in which R¹² and R⁷ are alkyl chains such as a C₁-C₆ straight or branched-chain alkyl.

Aminopyridines of the invention can be in a pharmaceutically acceptable salt form or as the free base. Suitable routes of administration include oral, buccal, topical (including trans-dermal) etc. Each agent is preferably administered by the oral route.

The effective dosage of each agent can readily be determined by a skilled person, having regard to typical factors each as the age, weight, sex and clinical history of the patient. A typical dosage of 3,4-DAP is 5 mg/kg to 100 mg/kg administered one to three times daily.

A pharmaceutical composition containing each active ingredient may be in a form suitable for oral use, for example, as tablets, troches, lozenges, aqueous or oily suspensions, dispersible powders or granules, emulsions, hard or soft capsules, or syrups or elixirs. Compositions intended for oral use may be prepared according to any method known in the art for the manufacture of pharmaceutical compositions and such compositions may contain one or more agents selected from sweetening agents, flavoring agents, coloring agents and preserving agents, in order to provide pharmaceutically elegant and palatable preparations. Tablets contain the active ingredient in admixture with non-toxic pharmaceutically acceptable excipients which are suitable for the manufacture of tablets. These excipients may be for example, inert diluents, such as calcium carbonate, sodium carbonate, lactose, calcium phosphate or sodium phosphate; granulating and disintegrating agents, for example corn starch, or alginic acid; binding agents, for example starch, gelatin or acacia, and lubricating agents, for example magnesium stearate, stearic acid or talc. The tablets may be uncoated or they may be coated by known techniques to delay disintegration and absorption in the gastrointestinal tract and thereby provide a sustained action over a longer period. For example, a time delay material such as glyceryl monostearate or glyceryl distearate may be employed. They may also be coated by the techniques described in U.S. Pat. Nos. 4,684,516, 4,775,536 and 4,265,874, to form osmotic therapeutic tablets for control release.

Formulations for oral use may also be presented as hard gelatin capsules in which the active ingredient is mixed with an inert solid diluent, for example calcium carbonate, calcium phosphate or kaolin, or as soft gelatin capsules in which the active ingredient is mixed with water or an oil medium, for example peanut oil, liquid paraffin or olive oil.

An alternative oral formulation, where control of gastrointestinal tract hydrolysis of the aminopyridine compound is sought, can be achieved using a controlled-release formulation, where the aminopyridine of the invention is encapsulated in an enteric coating.

Aqueous suspensions contain the active materials in admixture with excipients suitable for the manufacture of aqueous suspensions. Such excipients are suspending agents, for example sodium carboxymethylcellulose, methylcellulose, hydroxypropylmethylcellulose, sodium alginate, polyvinyl-pyrrolidone, gum tragacanth and gum acacia; dispersing or wetting agents such as a naturally occurring phosphatide, for example lecithin, or condensation products of an alkylene oxide with fatty acids, for example polyoxyethylene stearate, or condensation products of ethylene oxide with long chain aliphatic alcohols, for example heptadecaethyleneoxycetanol, or condensation products of ethylene oxide with partial esters derived from fatty acids and a hexitol such a polyoxyethylene with partial esters derived from fatty acids and hexitol anhydrides, for example polyoxyethylene sorbitan monooleate. The aqueous suspensions may also contain one or more preservatives, for example ethyl, or n-propyl p-hydroxybenzoate, one or more coloring agents, one or more flavoring agents, and one or more sweetening agents, such as sucrose or saccharin.

Oily suspensions may be formulated by suspending the active ingredient in a vegetable oil, for example arachis oil, olive oil, sesame oil or coconut oil, or in a mineral oil such as liquid paraffin. The oily suspensions may contain a thickening agent, for example beeswax, hard paraffin or cetyl alcohol. Sweetening agents such as those set forth above, and flavoring agents may be added to provide a palatable oral preparation. These compositions may be preserved by the addition of an anti-oxidant such as ascorbic acid.

Dispersible powders and granules suitable for preparation of an aqueous suspension by the addition of water provide the active ingredient in admixture with a dispersing or wetting agent, suspending agent and one or more preservatives. Suitable dispersing or wetting agents and suspending agents are exemplified, for example sweetening, flavoring and coloring agents, may also be present.

The pharmaceutical compositions of the invention may also be in the form of oil-in-water emulsions. The oily phase may be a vegetable oil, for example olive oil or arachis oil, or a mineral oil, for example liquid paraffin or mixtures of these. Suitable emulsifying agents may be naturally-occurring gums, for example gum acacia or gum tragacanth, naturally occurring phosphatides, for example soya bean, lecithin, and esters or partial esters derived from fatty acids and hexitol anhydrides, for example sorbitan monooleate and condensation products of the said partial esters with ethylene oxide, for example polyoxyethylene sorbitan monooleate. The emulsions may also contain sweetening and flavoring agents.

Syrups and elixirs may be formulated with sweetening agents, for example glycerol, propylene glycol, sorbitol or sucrose. Such formulations may also contain a demulcent, a preservative and flavoring and coloring agents. The pharmaceutical compositions may be in the form of a sterile injectable aqueous or oleaginous suspension. This suspension may be formulated according to the known art using those suitable dispersing or wetting agents and suspending agents which have been mentioned above. The sterile injectable preparation may also be in a sterile injectable solution or suspension in a non-toxic parenterally acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium. For this purpose any bland fixed oil may be employed including synthetic mono- or di-glycerides. In addition, fatty acids such as oleic acid find use in the preparation of injectables.

Each active agent, including the aminopyridine compound, may also be administered in the form of suppositories for rectal administration of the drug. These compositions can be prepared by mixing the drug with a suitable non-irritating excipient which is solid at ordinary temperatures but liquid at the rectal temperature and will therefore melt in the rectum to release the drug. Examples of such materials are cocoa butter and polyethylene glycols.

For topical use, creams, ointments, jellies, solutions or suspensions are suitable. Topical application includes the use of mouth washes and gargles.

### Incorporation by Reference

References and citations to other documents, such as patents, patent applications, patent publications, journals, books, papers, web contents, have been made throughout this disclosure. All such documents are hereby incorporated herein by reference in their entirety for all purposes.

### Equivalents

Various modifications of the invention and many further embodiments thereof, in addition to those shown and described herein, will become apparent to those skilled in the art from the full contents of this document, including references to the scientific and patent literature cited herein. The subject matter herein contains important information, exemplification and guidance that can be adapted to the practice of this invention in its various embodiments and equivalents thereof.

### EXAMPLES

### Example 1

### Aminopyridine Compounds that do not Accumulate in the Brain

The human pharmacokinetics for a set of 15 aminopyridines were modeled.

The pharmacokinetics of aminopyridine derivatives in humans and animals is satisfactorily modeled by means of physiologically-based pharmacokinetic (PBPK) modeling using simple ADME-related and physicochemical properties of the compounds (such as the extent of binding to plasma proteins, rates of irreversible metabolism, octanol/water partition coefficient and fractional charges at physiological pHs).

The values of many important properties for modeling, such as plasma protein binding and intestinal permeability, are obtained with sufficient reliability on the basis of octanol/water partition coefficient and fractional charges at physiological pH's.

The plasma selectivity of the aminopyridines were evaluated by modeling the brain AUC and the plasma AUC resulting from administration of a compound of the invention and comparing it to a modeled brain AUC and plasma AUC resulting from an administration of the active aminopyridine, i.e., in the cleaved form. The modeled plasma selectivity of an aminopyridine of the invention is represented by A:

$$A = \frac{( AUCbrain / AUCplasma ) uncleaved}{( AUCbrain / AUCplasma ) cleaved}$$

A was modeled for selected aminopyridines including a cleavable functional group. The pharmacokinetic program CLOE, produced by Cyprotex (Macclesfield, Cheshire, U.K.), was used. For each of the aminopyridines chosen, the modeling was performed on the aminopyridine compound including a cleavable functional group, as well as on the active aminopyridine as if administered in the active form without the cleavable functional group. For each model, an in silico determination was made for three physiochemical properties of that aminopyridine: cLogP, PSA and pKa.

Three pharmacokinetic parameters were considered: extra-hepatic hydrolysis (e.g., hydrolysis in the gastrointestinal tract), hepatic hydrolysis and hepatic inactivation (glucuronidation). For each aminopyridine modeled, modeling was performed assuming one of four different rates for each of these parameters. For each aminopyridine modeled, gastrointestinal hydrolysis was assumed to be either zero, slow (0.001/min), medium (0.01/min), or fast (0.1/min) while hepatic hydrolysis and hepatic inactivation were independently assumed to be zero, slow (0.1/min), medium (1.0/min) and fast (10.0/min).

Given rates for the three scenarios and values for cLogP, PSA, and pKa, the program CLOE modeled a resulting venous plasma AUC and brain interstitial AUC. For each aminopyridine compound modeled in this way, CLOE was also used to model a resulting plasma and brain AUC for the corresponding active aminopyridine as if administered without the cleavable functional group.

The program was used to model the pharmacokinetic properties of the 15 aminopyridines represented by formulas (XI), (XII), (XIII), (XIV), (XV), (XVI), (XVII), (XVIII), (XXIV), (XXV), (XXVI), (XXVII), (LIV), (XXVIII), and (XXXIII):

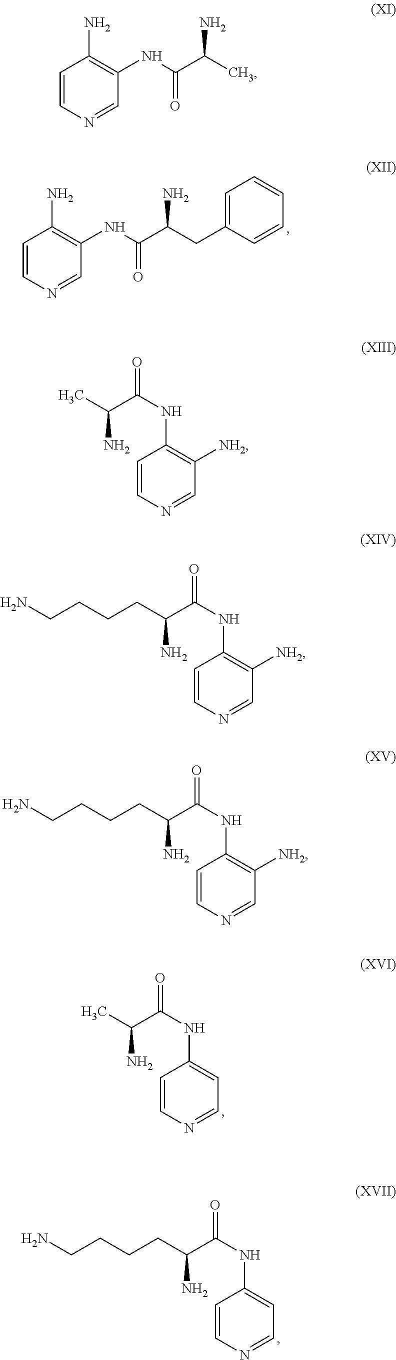

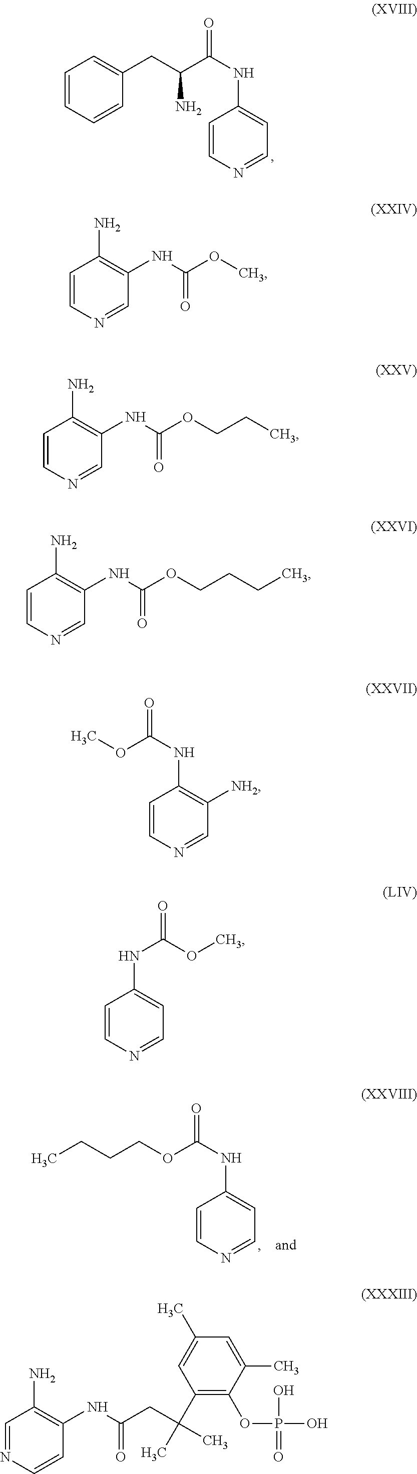

According to the modeling, the plasma and brain concentrations of the free aminopyridine is highly dependent on the on the rate of extra-hepatic hydrolysis, i.e., hydrolysis in the gastrointestinal tract. The rate of liver hydrolysis of the compound to give the active aminopyridine is dependent on the rate of penetration of the compound into the liver. The rate of penetration into the liver is controlled by the aminopyridine's octanol/water partition coefficient (LogP), as obtained from the compound's LogP and pKa values. The rate of hepatic inactivation of the compound through glucuronidation follows the same requirements as for hepatic hydrolysis.

For the 15 selected aminopyridines show above, the CLOE modeling demonstrated that the rate of extra-hepatic hydrolysis is a highly important factor for determining plasma and brain levels of the free active aminopyridines. The modeled plasma selectivity of an aminopyridine of the invention is represented by A. Aminopyridines with A>1 exhibit greater selectivity for plasma versus brain. Aminopyridines with A=1 do not exhibit such plasma selectivity.

For aminopyridine compounds that undergo fast or medium extra-hepatic hydrolysis in the GI tract, the modeling showed the plasma and brain AUC of the active aminopyridine resulting from administering the uncleaved compound to be very similar to the plasma and brain AUC of the active aminopyridine resulting from administering the cleaved aminopyridine directly (both administered orally), i.e. A=1. Similarly, for aminopyridine compounds that undergo zero hydrolysis in the gastrointestinal tract, there was no difference observed between the plasma and brain AUC of the active aminopyridine resulting from administering the uncleaved compound and the plasma and brain AUC of the active aminopyridine resulting from administering the cleaved aminopyridine directly (A=1). These observations were shown to be independent of the rates of hepatic hydrolysis or hepatic inactivation.

However, for slow rates of extra-hepatic hydrolysis of the compounds, the modeling identified carbamate aminopyridines that show an increased selectivity for plasma over brain, i.e. A>1, as represented by formulas (XXV), (XXVI), (XXVII), and (LIV) in Table 1. Table 2 shows the raw data from the CLOE modeling.

TABLE 1
Carbamate aminopyridines with A > 1

| Formula | Structure | pKa | logP | A |
|---|---|---|---|---|
| (XXV) (family 10) | 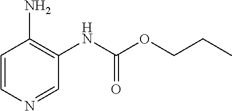 | 8.2 | 1.7 | 1.13 |
| (XXVI) (family 11) | 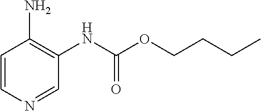 | 8.2 | 2.3 | 2.02 |
| (XXVII) (family 12) | 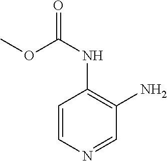 | 5.4 | 0.8 | 1.21 |
| (LIV) (family 13) | 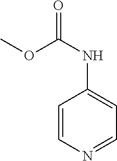 | 4.9 | 1.3 | 1.40 |

TABLE 2
Raw data from CLOE modeling (columns labeled “prodrug” refer to aminopyridines administered
in the uncleaved form; columns labeled “active” refer to aminopyridines administered in the cleaved form)

| | scenario and rate | | | venous plasma | | brain interstitial | | ratios | | |
|---|---|---|---|---|---|---|---|---|---|---|
| | gut | hepatic | hepatic | AUC (min * kg/ml) | | AUC (min * kg/ml) | | AUC plasma/AUC brain | | |
| family | hydrolysis | hydrolysis | inactivation | prodrug | active | prodrug | active | prodrug | active | ratio (I/J) |
| 10 | slow | fast | zero | 0.035 | 0.612 | 0.030 | 0.595 | 1.166 | 1.028 | 1.135 |
| 10 | slow | medium | zero | 0.034 | 0.612 | 0.030 | 0.595 | 1.166 | 1.028 | 1.134 |
| 10 | slow | slow | zero | 0.034 | 0.612 | 0.029 | 0.595 | 1.165 | 1.028 | 1.133 |
| 11 | slow | fast | zero | 0.082 | 0.612 | 0.040 | 0.595 | 2.078 | 1.028 | 2.021 |
| 11 | slow | medium | zero | 0.082 | 0.612 | 0.040 | 0.595 | 2.076 | 1.028 | 2.020 |
| 11 | slow | slow | zero | 0.081 | 0.612 | 0.039 | 0.595 | 2.056 | 1.028 | 2.000 |
| 12 | slow | fast | zero | 0.034 | 0.612 | 0.027 | 0.595 | 1.241 | 1.028 | 1.207 |
| 12 | slow | medium | zero | 0.033 | 0.612 | 0.027 | 0.595 | 1.240 | 1.028 | 1.206 |
| 12 | slow | slow | zero | 0.033 | 0.612 | 0.027 | 0.595 | 1.233 | 1.028 | 1.200 |
| 13 | slow | fast | zero | 0.116 | 0.638 | 0.077 | 0.596 | 1.502 | 1.072 | 1.401 |
| 13 | slow | medium | zero | 0.115 | 0.638 | 0.077 | 0.596 | 1.498 | 1.072 | 1.398 |
| 13 | slow | slow | zero | 0.106 | 0.638 | 0.072 | 0.596 | 1.466 | 1.072 | 1.368 |

The CLOE modeling data indicate that a key balance of pKa and logP is important to optimize hepatic penetration. By exploiting this physicochemical balance, and synthesizing aminopyridines with low susceptibility to gastrointestinal hydrolysis (e.g., the carbamates shown in Table 1), plasma selectivity is attainable. The compounds of the invention, such as the carbamate aminopyridines, fulfill the requirement of slow rates of gastrointestinal hydrolysis.

1. An aminopyridine or a pharmaceutically-acceptable salt thereof, wherein the aminopyridine is represented by formula (I): 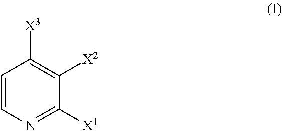
wherein:
X¹ is selected from H, NH₂, NHR¹, N═NR², N═R³, or NHCOOCH₃,
X² is selected from H, NH₂, NHR¹, N═NR², or N═R³,
X³ is selected from NH₂, NHR¹, N═NR², or N═CR³, wherein X² and X³ are not both NH₂;
R¹ is selected from:
COR⁴, wherein R⁴ is an amino acid attached at the carbonyl carbon;
COOR⁵, wherein R⁵ is a C₂ or higher alkyl group;
COR⁶, wherein R⁶ is a branched chain alkyl optionally substituted with a phenyl group, wherein the phenyl group is optionally substituted with a phosphonooxy group and optionally substituted with one or more alkyl groups;
a pyrone; and
SO₃Na;
R² is a heterocycloalkenyl optionally substituted with one or more amino groups;
R³ is a phenyl group optionally substituted with one or more halogens; or
R³ is selected from the group consisting of formulas (II), (III), (IV), (V), (VI), (VII), (VIII), and (IX): 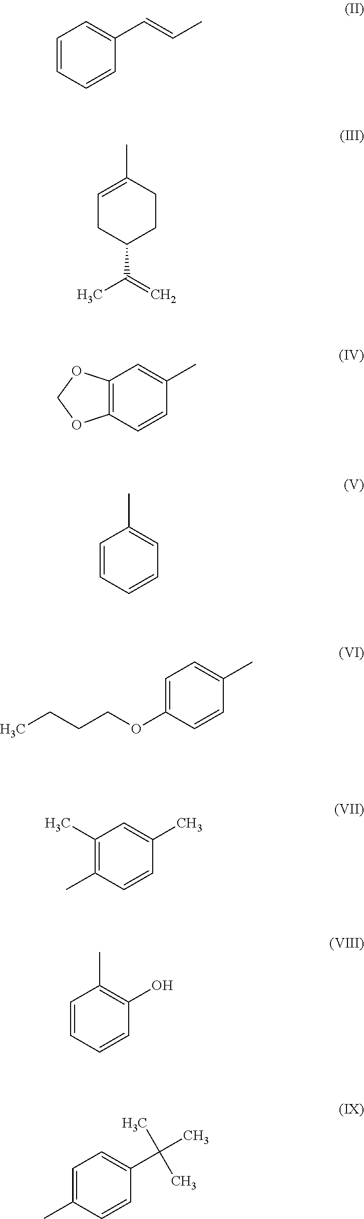
with the proviso that when X³ is COOR⁵, R⁵ is not an ethyl group, tert-butyl group, or n-dodecyl group.

2. The aminopyridine of claim 1, wherein R⁴ is alanine, lysine, or phenylalanine.

3. The aminopyridine of claim 2, represented by a formula selected from the group consisting of (X), (XI), (XII), (XIII), (XIV), (XV), (XVI), (XVII), and (XVIII): 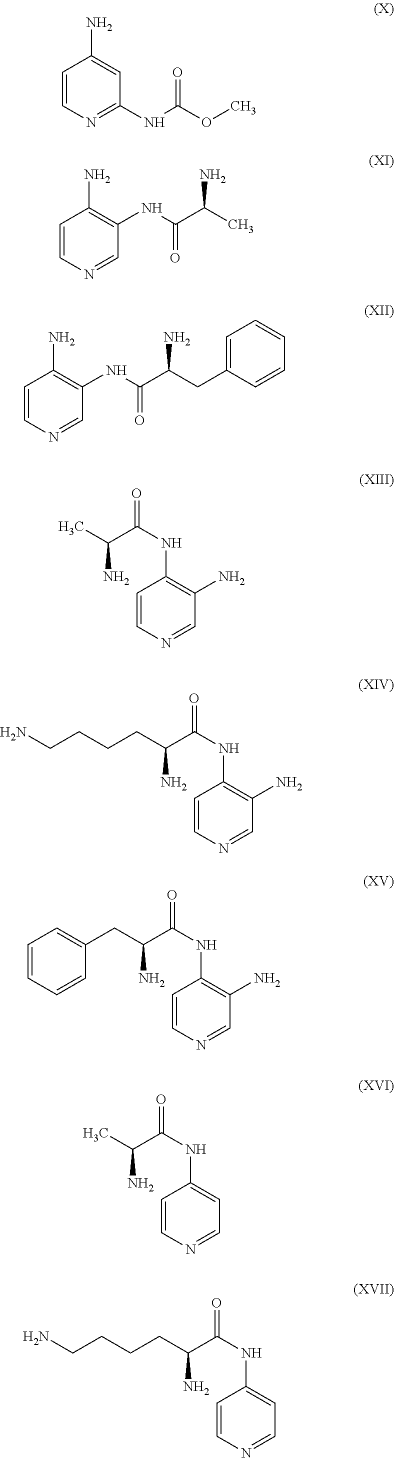 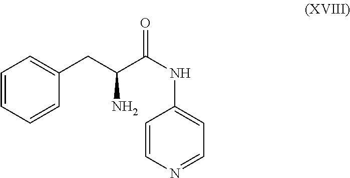 [SMILES: N[C@@H](Cc1ccccc1)C(=O)Nc1ccncc1]
or a pharmaceutically acceptable salt thereof.

4. The aminopyridine of claim 1, represented by formula (XIX): 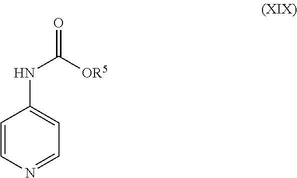
wherein R⁵ is an n-propyl group; an n-butyl group; a sec-butyl group, or a straight or branched C₅ or higher alkyl chain, with the proviso that R⁵ is not an n-dodecyl group;
or a pharmaceutically acceptable salt thereof.

5. The aminopyridine of claim 1, represented by a formula selected from the group consisting of (XX), (XXI), (XXII), and (XXIII): 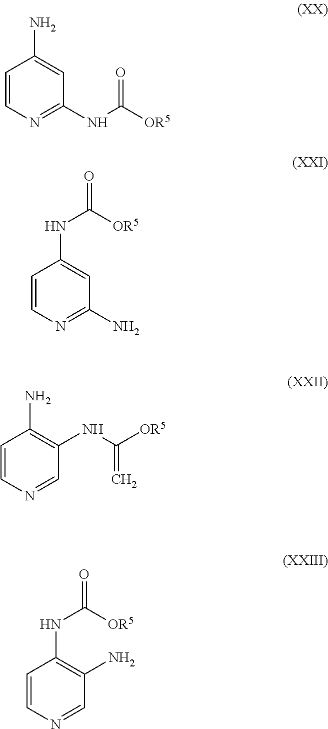
wherein R⁵ is a C₂ or higher alkyl group; or
or a pharmaceutically acceptable salt thereof.

6. The aminopyridine of claim 5, represented by formula (XXV): 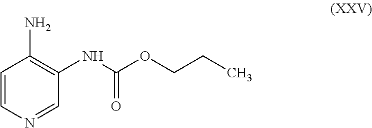
or a pharmaceutically acceptable salt thereof.

7. The aminopyridine of claim 5, represented by formula (XXVI): 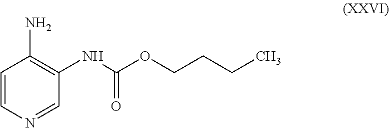
or a pharmaceutically acceptable salt thereof.

8. The aminopyridine of claim 5, represented by formula (XXVII): 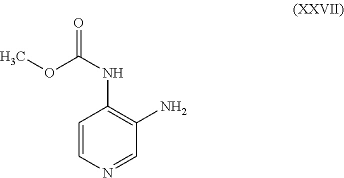
or a pharmaceutically acceptable salt thereof.

9. The aminopyridine of claim 5, represented by formula (XXVIII): 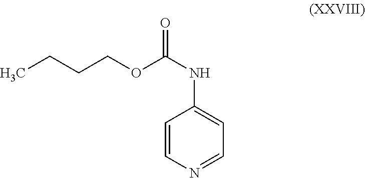
or a pharmaceutically acceptable salt thereof.

10. The aminopyridine of claim 1, represented by the a formula selected from the group consisting of (XXIX), (XXX), and (XXXI): 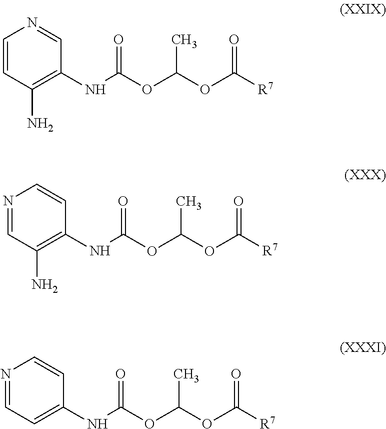
wherein R⁷ is a alkyl chain;
or a pharmaceutically acceptable salt thereof.

11. The aminopyridine of claim 10, wherein R⁷ is a C₁-C₆ straight or branched-chain alkyl.

12. The aminopyridine of claim 1, wherein R⁶ is represented by formula (XXXII): 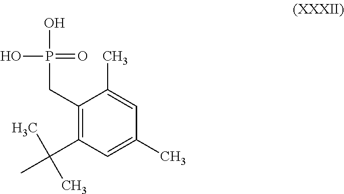 [SMILES: Cc1cc(C)c(CP(=O)(O)O)c(C(C)(C)C)c1]
or a pharmaceutically acceptable salt thereof.

13. The aminopyridine of claim 15 represented by the formula (XXXIII): 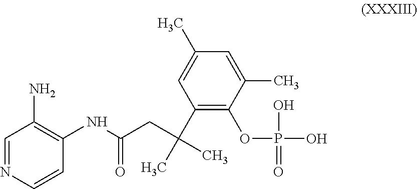 [SMILES: Cc1cc(C)c(OP(=O)(O)O)c(C(C)(C)CC(=O)Nc2ccncc2N)c1]
or a pharmaceutically acceptable salt thereof.

14. The aminopyridine of claim 1, wherein:
X³ is NH₂,
X¹ is H, and
X² is N═R³.

15. The aminopyridine of claim 14, represented by formula (XXXIV): 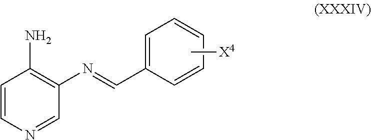
wherein X⁴ is a halogen;
or a pharmaceutically acceptable salt thereof.

16. The aminopyridine of claim 1, represented by formula (XXXV): 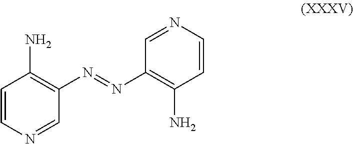 [SMILES: Nc1ccncc1/N=N/c1cnccc1N]
or a pharmaceutically acceptable salt thereof.

17. The aminopyridine of claim 1, represented by formula (XXXVI): 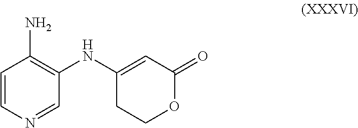 [SMILES: Nc1ccncc1NC1=CC(=O)OCC1]
or a pharmaceutically acceptable salt thereof.

18. The aminopyridine of claim 1, represented by formula (XXXVII): 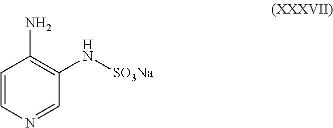
or a pharmaceutically acceptable salt thereof.

19. A method of treating a neural disorder, the method comprising administering an effective dose of an aminopyridine, or a pharmaceutically acceptable salt thereof, wherein the aminopyridine is represented by formula (I): 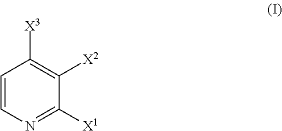
wherein:
X¹ is selected from H, NH₂, NHR¹, N═NR², N═R³, or NHCOOCH₃,
X² is selected from H, NH₂, NHR¹, N═NR², or N═R³,
X³ is selected from NH₂, NHR¹, N═NR², or N═CR³, wherein X² and X³ are not both NH₂;
R¹ is selected from:
COR⁴, wherein R⁴ is an amino acid attached at the carbonyl carbon;
COOR⁵, wherein R⁵ is a C₂ or higher alkyl group;
COR⁶, wherein R⁶ is a branched chain alkyl optionally substituted with a phenyl group, wherein the phenyl group is optionally substituted with a phosphonooxy group and optionally substituted with one or more alkyl groups;
a pyrone; and
SO₃Na;
R² is a heterocycloalkenyl optionally substituted with one or more amino groups;
R³ is a phenyl group optionally substituted with one or more halogens; or
R³ is selected from the group consisting of formulas (II), (III), (IV), (V), (VI), (VII), (VIII), and (IX): 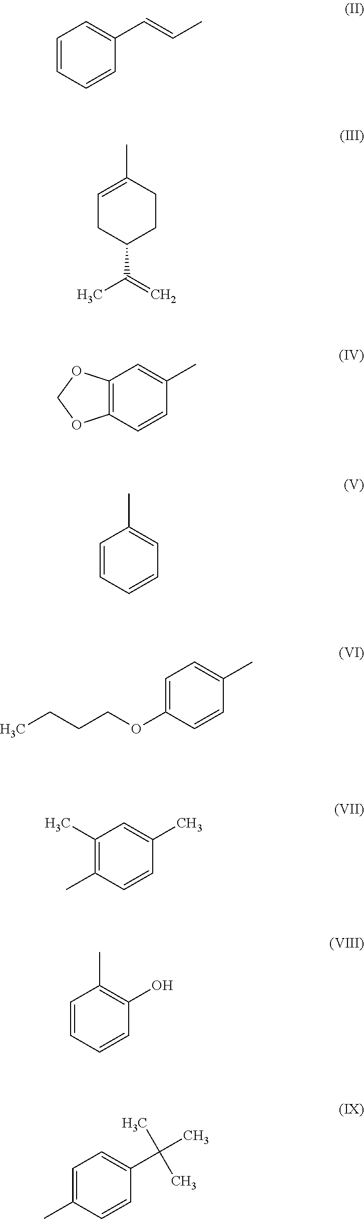
with the proviso that when X³ is COOR⁵, R⁵ is not an ethyl group, tert-butyl group, or n-dodecyl group.

20. The method of claim 19, wherein R⁴ is alanine, lysine, or phenylalanine.

21. The method of claim 20, wherein the aminopyridine is represented by a formula selected from the group consisting of (XI), (XII), (XIII), (XIV), (XV), (XVI), (XVII), and (XVIII): 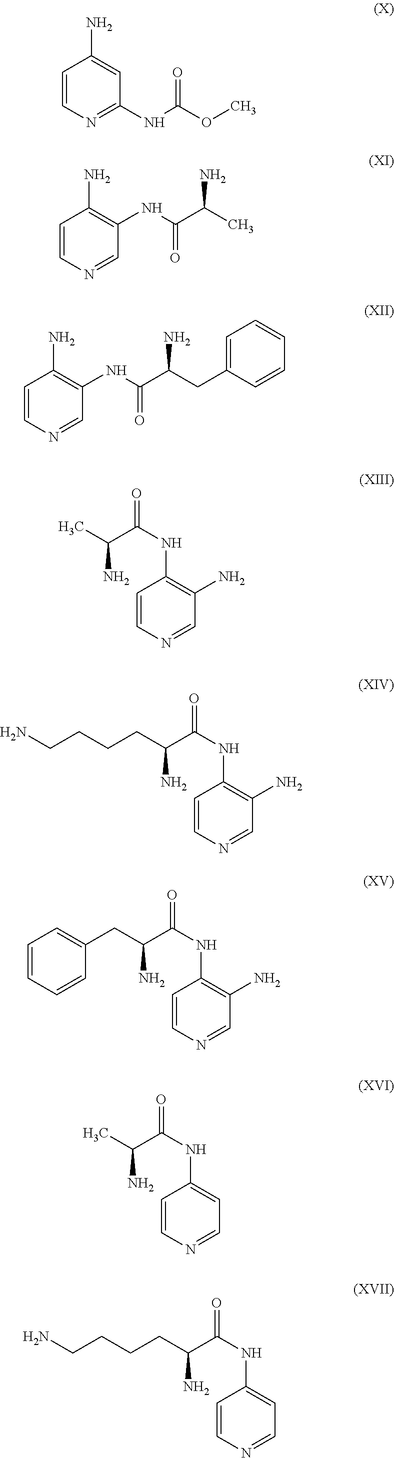 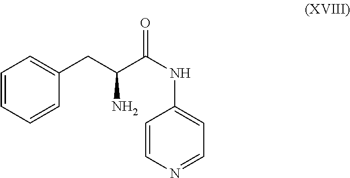 [SMILES: N[C@@H](Cc1ccccc1)C(=O)Nc1ccncc1]
or a pharmaceutically acceptable salt thereof.

22. The method of claim 19, wherein the aminopyridine is represented by formula (XIX): 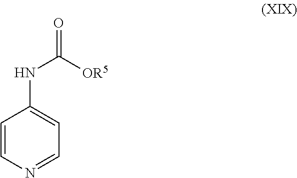
wherein R⁵ is an n-propyl group; an n-butyl group; a sec-butyl group, or a straight or branched C₅ or higher alkyl chain, with the proviso that R⁵ is not an n-dodecyl group;
or a pharmaceutically acceptable salt thereof.

23. The method of claim 19, wherein the aminopyridine is represented by a formula selected from the group consisting of (XX), (XXI), (XXII), and (XXIII): 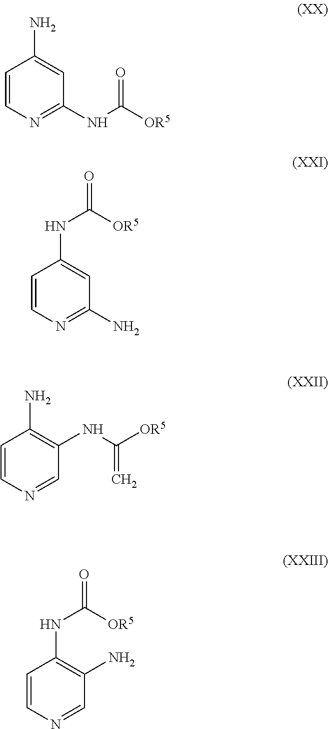
wherein R⁵ is a C₂ or higher alkyl group; or
a pharmaceutically acceptable salt thereof.

24. The method of claim 23, wherein the aminopyridine is represented by formula (XXV): 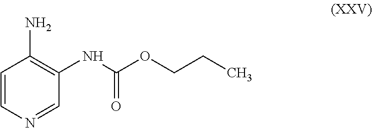
or a pharmaceutically acceptable salt thereof.

25. The method of claim 23, wherein the aminopyridine is represented by formula (XXVI): 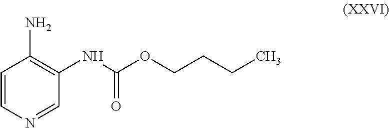
or a pharmaceutically acceptable salt thereof.

26. The method of claim 23, wherein the aminopyridine is represented by formula (XXVII): 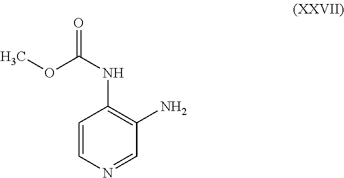
or a pharmaceutically acceptable salt thereof.

27. The method of claim 23, wherein the aminopyridine is represented by formula (XXVIII): 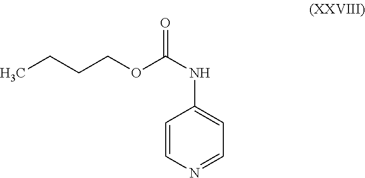
or a pharmaceutically acceptable salt thereof.

28. The method of claim 19, wherein the aminopyridine is represented by the a formula selected from the group consisting of (XXIX), (XXX), and (XXXI): 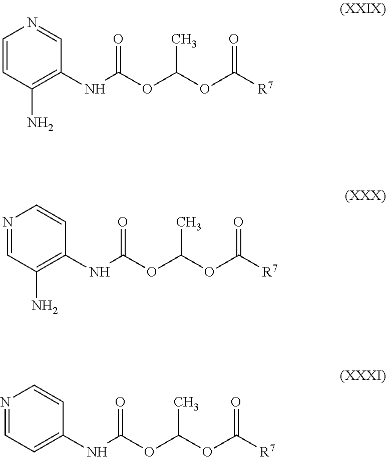
wherein R⁷ is a alkyl chain;
or a pharmaceutically acceptable salt thereof.

29. The method of claim 28, wherein R⁷ is a C₁-C₆ straight or branched-chain alkyl.

30. The method of claim 19, wherein R⁶ is represented by formula (XXXII): 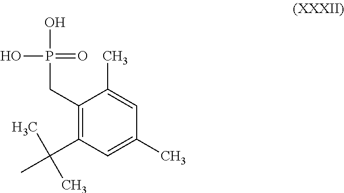 [SMILES: Cc1cc(C)c(CP(=O)(O)O)c(C(C)(C)C)c1]
or a pharmaceutically acceptable salt thereof.

31. The method of claim 30, wherein the aminopyridine is represented by the formula (XXXIII): 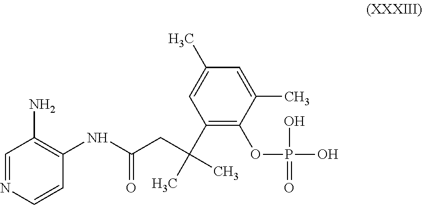 [SMILES: Cc1cc(C)c(OP(=O)(O)O)c(C(C)(C)CC(=O)Nc2ccncc2N)c1]
or a pharmaceutically acceptable salt thereof.

32. The method of claim 19, wherein:
X³ is NH₂,
X¹ is H, and
X² is N═R³.

33. The method of claim 32, wherein the aminopyridine is represented by formula (XXXIV): 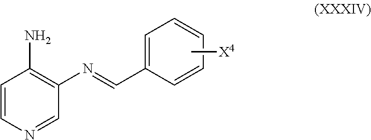
wherein X⁴ is a halogen;
or a pharmaceutically acceptable salt thereof.

34. The method of claim 19, wherein the aminopyridine is represented by formula (XXXV): 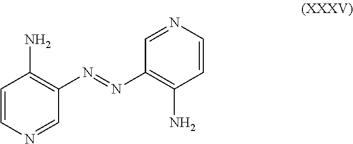 [SMILES: Nc1ccncc1/N=N/c1cnccc1N]
or a pharmaceutically acceptable salt thereof.

35. The method of claim 19, wherein the aminopyridine is represented by formula (XXXVI): 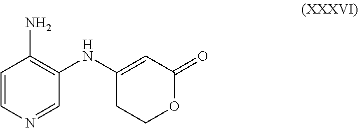 [SMILES: Nc1ccncc1NC1=CC(=O)OCC1]
or a pharmaceutically acceptable salt thereof.

36. The method of claim 19, wherein the aminopyridine is represented by formula (XXXVII): 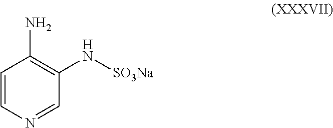
or a pharmaceutically acceptable salt thereof.
